(19) United States
(12) Patent Application Publication
(10) Pub. No.: US 2021/0228462 A1
(43) Pub. Date: Jul. 29, 2021

(54) COMPOSITION FOR DYEING

(71) Applicant: LG HOUSEHOLD & HEALTH CARE LTD., Seoul (KR)

(72) Inventors: Jun Seok YEOM, Daejeon (KR); Seong Cheon LEE, Daejeon (KR); Eu Gene HUR, Daejeon (KR); Yoon Kwan KIM, Daejeon (KR); Seol Hoon LEE, Daejeon (KR); Nae Gyu KANG, Daejeon (KR)

(73) Assignee: LG HOUSEHOLD & HEALTH CARE LTD., Seoul (KR)

(21) Appl. No.: 16/972,431

(22) PCT Filed: Jun. 7, 2018
(86) PCT No.: PCT/KR2018/006450

§ 371 Date: Dec. 4, 2020

(51) Int. Cl. A61K 8/49; A61K 8/02; A61K 8/34; A61K 8/90; A61K 8/81; A61K 8/31; A61K 8/92; A61K 8/35; A61K 8/11; A61Q 1/04

(52) U.S. Cl. CPC A61K 8/492; A61K 8/0245; A61K 8/345; A61K 8/90; A61K 8/8117; A61K 8/496; A61K 2800/412; A61K 8/8111; A61K 8/925; A61K 8/355; A61K 8/11; A61Q 1/04; A61K 2800/432; A61K 8/31

(57) ABSTRACT

The present invention relates to a composition for dyeing. The composition for dyeing according to the present invention forms a covalent bond without damaging skin, hair, nails, fibers, or leather by using a reaction promoting additive for promoting peptide bond formation in addition to a carbodiimide-based compound, thereby providing a desired semi-permanent dyeing effect.

### TECHNICAL FIELD

The present invention relates to a dyeing composition.

### BACKGROUND ART

Generally, a hair dye may be classified into a permanent hair dye (oxidative hair dye), a semi-permanent hair dye (non-oxidative hair dye) and a temporary colorant according to the durability of a dyed color and the characteristics of a composition. In the case of an oxidative hair dye which is the most widely used type of hair dye, a low-molecular-weight oxidative dye or pigment causes oxidative polymerization in hair, thereby forming colors and obtaining various colors, and thus has excellent hair dyeing ability. Such an oxidative hair dye composition contains one or more types of dyes or dye precursors and one or more types of couplers which form a dye or pigment by being oxidized by an oxidizing agent, and the oxidative hair dye composition is used by being mixed with an oxidizing agent consisting of a diluted hydrogen peroxide solution immediately before application to hair. The purpose of using such a hair dye is to dye hair as completely as possible and to sustain a hair dyeing effect for a long time. In addition, the non-oxidative hair dye is a hair dye that is useful for providing a natural color close to the primary color to hair, causes relatively less damage to hair and is able to simply dye at the same time as a perm, and thus its availability is gradually expanding. However, hair dyes currently on the market are easily discolored due to fading caused by repeated shampooing when dyed in brighter colors, and particularly, oxidative hair dyes have a disadvantage of worsening hair texture and reducing hair gloss compared with healthy hair since disulfide bonds in the hair are destroyed during hair bleaching by the action of an oxidizing agent such as hydrogen peroxide, causing severe damage to the hair.

In the case of nail polishes, since a dye is not bonded by itself, nail polishing is performed by forming a color layer on the surface of a nail using an adhesive such as nitrocellulose lacquer or cyanoacrylate. These compounds may damage a nail surface and form a thick pigment layer, so there are limitations in color expression.

Skin coloring is for coloring skin by color make-up in the way of leaving a powder or a component such as iron oxide on the skin surface, or by deposition of a pigment such as a tint on the skin, but has low durability, and a method like tattooing is for inserting a pigment into the skin, causing side effects such as inflammation.

In order to improve the above-described shortcoming, recently, a method of chemically modifying the surfaces of the skin and hair with a cosmetic material using a bioconjugation method has begun to be introduced.

While L'Oreal has recently suggested a method of quickly obtaining an artificial tanning effect of dihydroxyacetone (DHA) by modifying the skin with an amine group-introduced alkoxysilane (US Patent Application No. 2010-516546), due to high reactivity, the alkoxysilane is classified as Class 3 in MSDS toxicity, and thus is difficult to be applied in actual products. In previous research disclosed in US Patent Publication No. 2011-0274639 and US Patent Publication No. 2006-0233729, methods of modifying skin were also proposed, but the technology of applying a bioconjugation method to cosmetics is in its early stage so that there are not many examples yet.

The present inventors have invented a method of modifying a dermal protein with an amine group, a carboxyl group, a thiol group or a hydroxyl group, which was disclosed in previous research. Particularly, they have found a method of directly modifying proteins present in the hair and skin using polymeric carbodiimide (PCI) (Korean Patent Publication No. 2008-0064467) and are conducting research on applying this method in personal care. Since acyl urea is formed by a carbodiimide reaction, causing toxicity in the human body, such a reaction may be minimized by using a polymer-type carbodiimide. However, since reaction accelerators such as PCI, which can apply the corresponding reaction to the skin, are not diverse, to apply the bioconjugation method universally, there is a need for reaction media that are safe for the human body and can remain in the skin for a long time.

### DISCLOSURE

### Technical Problem

The present invention is directed to providing a dyeing composition that is able to consistently impart a semi-permanent dyeing effect without damaging hair, skin, fingernails and toenails, fiber or leather.

### Technical Solution

The present invention provides a dyeing composition which includes a carbodiimide-based compound, a reaction accelerating additive and a dye.

### Advantageous Effects

A dyeing composition according to the present invention contains a carbodiimide-based compound, a reaction accelerating additive for forming peptide bonds and a dye, and thus can effectively form a covalent bond without damaging the surface of hair, skin, fingernails, toenails, fiber or leather, thus imparting a semi-permanent dyeing effect.

### DESCRIPTION OF DRAWINGS

FIG. 1 is a graph that shows the a* value (red) obtained as a result of treating pig skin with either the composition of Example 1 or Comparative Example 1.

### MODES OF THE INVENTION

The present invention relates to a dye composition, which includes a carbodiimide-based compound, a reaction accelerating additive and a dye.

As the carbodiimide-based compound and the reaction accelerating additive are reacted with, for example, a conventionally known dye, the dye composition according to the present invention can be safe for the surface of skin, hair, fiber or leather and the dye component can remain for a long time, resulting in a maximized dye effect.

The carbodiimide-based compound is a generic term for compounds containing a structure represented by the following Formula 1 in a molecule, and is referred to as PCI in the present invention.

N═C═N— [Formula 1]

The carbodiimide-based compound preferably includes at least approximately 1 to 1000 structures of Formula 1, more preferably include 1 to 100 structures represented by Formula 1 in the molecule, and still more preferably include 1 to 10 structures represented by Formula 1 in the molecule.

In this case, when there are more than 1000 carbodiimide groups represented by Formula 1 in one molecule, a raw material has a very high viscosity, a very large molecular weight and too many sites where the reaction occurs, and since a molecule containing carbodiimide groups becomes excessively larger, it hides a part exhibiting a functional effect of the molecule, which has a substantial function, causing reduced efficiency.

In the present invention, a specific example of the carbodiimide-based compound may be a compound represented by Formula 2 below, wherein the compound of Formula 2 in which n is 4 and m is 11 is referred to as benzene, 1,3-bis(1-isocyanato-1-methylethyl)-, homopolymer, polyethylene glycol mono-Me-ether-blocked (hereinafter, a polymer-type carbodiimide-based compound of Compound 2).

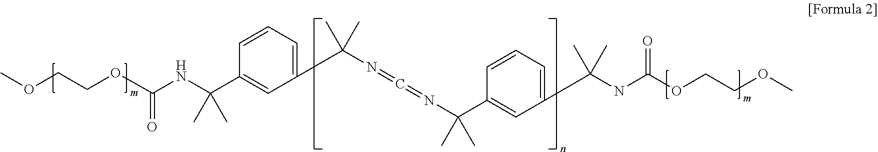

In Formula 2, n is 1 to 100, and m is 1 to 100.

The carbodiimide-based compound may be used at 0.001 to 10 parts by weight, 0.01 to 7 parts by weight or 0.1 to 5 parts by weight with respect to 100 parts by weight of the total composition. When the content is less than 0.001 parts by weight, it is difficult for the dye to sufficiently adhere to the surface of hair, skin, fingernails, toenails, fiber or leather, making it difficult to exhibit a continuous dyeing effect, whereas when the content is more than 10 parts by weight, due to the excessive amount of the carbodiimide-based compound, which is more than reaction sites present on the surface of hair, skin, fingernails, toenails, fiber or leather, it is difficult to improve the efficacy, odor and properties of a product, and there are problems in formulation and formulation stabilization.

The “dyeing effect” used herein refers to an ability of making a desired color more visible by changing a unique surface color of hair, skin, fingernails, toenails, fiber or leather using a dye or a colourant, and sustaining it on the surface semi-permanently or permanently.

The reaction accelerating additive for forming a peptide bond may be used at 0.0001 to 10 parts by weight, preferably 0.001 to 1 part by weight, and more preferably, 0.001 to 0.1 parts by weight with respect to with respect to 100 parts by weight of the total composition. When the additive is used at more than 10 parts by weight, since a chemical reaction speed is saturated, a product is not only uneconomical, but also not improved in stability. When the additive is used at less than 0.0001, there is no effect of adhering the dye to the surface of hair, skin, fingernails, toenails, fiber or leather.

The reaction accelerating additive for forming a peptide bond may be, specifically, one or more selected from the group consisting of hydroxysuccinimide, hydroxycarbamide, hydroxytriazole, hydroxybenzotriazole, hydroxypyridinone, hydroxyphenylbenzimidazole, hydroxyindolinone, hydroxybenzotriazine and oxyma, but the present invention is not limited thereto. In addition, the reaction accelerating additive may be a water-soluble nonionic polymer, a bead- or resin-type polymer resin, or combined with silica beads to increase stability and biosafety in the formulation.

The water-soluble nonionic polymer may have a weight average molecular weight of 1,000 Da or more and less than 300,000 Da, and when the molecular weight is 300,000 Da or more, it is difficult to exhibit efficacy because the proportion of the carbodiimide-based compound inducing a covalent bond is too small.

The water-soluble nonionic polymer may be, specifically, one or more polymers selected from the group consisting of polyethylene glycol, polyvinyl alcohol, polyethylene oxide, polyacrylonitrile, polyvinylpyrrolidone, polyisopropylacrylamide, a cellulose derivative, a starch derivative, dextran and guar gum.

It is preferable that the resin-type polymer preferably has a weight average molecular weight of 1,000 Da or more and less than 300,000 Da, and when a molecular weight is 300,000 Da or more, it is difficult to exhibit efficacy because there is a very small proportion of the carbodiimide-based compound inducing a covalent bond.

When the bead-type polymer resin (polymer bead) may have an average particle size of 100 nm to 1 mm, preferably 100 nm to 100 μm, more preferably, 1 to 100 μm, and most preferably, 1 to 70 μm. When the polymer bead has a diameter of less than 100 nm, there may be a problem in terms of safety for the surface of hair, skin, fingernails, toenails, fiber or leather, and when the polymer bead has a diameter of more than 1 mm, there are problems in terms of formulation and stabilization.

The bead- or resin-type polymer resin may be a copolymer polymerized with a monomer selected from the group consisting of, specifically, styrene, ethylene, butadiene, acrylonitrile, methyl styrene, terephthalate, ethylene chloride, ether ketone, ether imide, ether sulfone, phthalamide, phenylene ether, phenylene oxide, phenylene sulfide, sulfone, urethane, vinylidene fluoride and tetrafluoroethylene.

The silica bead may have an average particle size of 100 nm to 1 mm, preferably, 100 nm to 100 μm, more preferably, 1 to 100 μm, and most preferably 1 to 70 μm. When the diameter of the silica bead is smaller than 100 nm, there is a safety problem for the surface of hair, skin, fingernails, toenails, fiber or leather, and when the diameter of the silica bead is larger than 1 mm, there are problems in terms of formulation and stabilization.

In addition, the dye used in the present invention may include one or more atomic groups selected from the group consisting of —NO₂, —N═N—, C═O, C═C, C═N—, C═S, —N═O and —N═NO as a chromophore, but the present invention is not limited thereto. In addition, the dye may include one or more atomic groups selected from —OCH₃, —N(CH₃)₂, —NH(CH₃), —NO₂, —CF₃, —OH, —OCH₃, —Cl and —NH₂ as an auxochrome, but the present invention is not limited thereto. In addition, the dye may include one or more reaction groups selected from the group consisting of —COONa, —COOK, —COOH, —NH₂, —NHR, —NR₂, —Cl, —Br, —I and —F, but the present invention is not limited thereto. In addition, the dye may be a natural or synthetic dye used for hair, skin, fiber or leather, wherein the natural dye may be purpurin, munjistin, melanin or a melanin precursor, but the present invention is not limited thereto, and the synthetic dye may be one or more selected from the group consisting of Red No. 3, Red No. 104, Red No. 105, Red No. 201, Red No. 202, Red No. 220, Red No. 227, Red No. 230, Red No. 231, Red No. 232, Red No. 401, Red No. 405, Yellow No. 4, Yellow No. 202, Orange No. 207, Red No. 106, Red No. 213, Red No. 214, Red No. 215, Yellow No. 404, Yellow No. 405, Blue No. 403, Disperse Blue 1, Disperse Violet 1, Disperse Orange 3, Disperse Black 9, HC Blue 2, HC Red 3, HC Yellow 5 and HC Red 1, but the present invention is not limited thereto.

The dye may be used at 0.001 to 50 parts by weight, 0.001 to 30 parts by weight, 0.01 to 10 parts by weight or 0.1 to 5 parts by weight with respect to 100 parts by weight of the total composition. When the content is less than 0.001 parts by weight, there is a limitation in effective performance, and when the content is more than 50 parts by weight, there are problems in terms of formulation and formulation stabilization.

Particularly, in the present invention, the dye may include a carboxyl group or amine group in the molecule, thereby improving reaction efficiency with the carbodiimide-based compound.

In addition, the dye of the present invention may have a functional group which makes a covalent bond with a protein residue on the surface of hair, skin, fingernails, toenails, fiber or leather.

The functional group which makes a covalent bond with a protein residue may be one or more selected from the group consisting of carbodiimide, imidoester, arylazide, diazirine, hydroxymethyl phosphine, pentafluorophenylester, pyridyl disulfide, sulfo-hydroxysuccinimide ester, alkoxyamine, hydrazide, haloacetyl, azide, carbonate, aldehyde, propionaldehyde, butylaldehyde, nitrophenyl carbonate, aziridine, isocyanate, thiocyanate, epoxide, trecylate, succinimide, hydroxysuccinimidyl ester, imidazole, oxycarbonyl imidazole, imine, thiol, maleimide, vinylsulfone, ethyleneimide, thioether, acrylonitrile, acrylic or methacrylic ester, disulfide, ketone, carboxylic acid and phosphate, but the present invention is not limited thereto.

In addition, the protein may include a reactive residue such as thiol, hydroxyl, carboxyl or amine.

As an exemplary embodiment of the dyeing composition according to the present invention, a dye with a carboxyl group is formed in the form of a reactive ester dye directly having bioreactivity, or the form that is targeted by a dye with an amine group by reactive esterification of an amino acid (e.g., aspartic acid or glutamic acid) excessively present in hair (17.5 to 21.9%) or dermal keratin (15.5 to 23.5%), thereby improving reactive efficiency, and thus apparently improving its effect. As a more preferable exemplary embodiment, first, a polymer-type carbodiimide-based compound may have a primary reaction on the surface of a dye component molecule or protein with a carboxyl group to form a reactive ester, the reactive ester may be substituted with a reaction accelerating additive to increase selectivity, and then a dye having an amine in the molecule may be reacted again with the formed reactive ester functional group or with an amine on the skin surface, thereby obtaining an excellent dyeing effect.

The following Reaction Scheme 1 shows a schematic diagram of a reaction of forming a reactive ester by a primary reaction between a dye molecule having a carboxyl group and a carbodiimide-based compound, substituting the reactive ester with a reaction accelerating additive to increase selectivity, and forming a covalent bond with an amino acid having an amine group in the body.

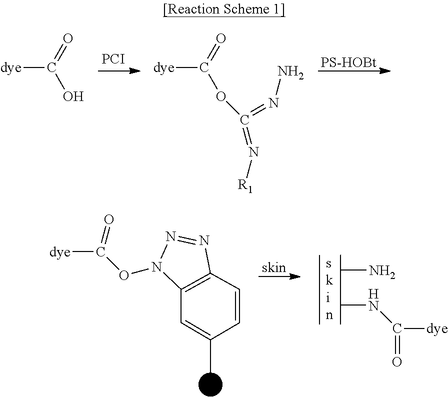

The following Reaction Scheme 2 shows a schematic diagram of a reaction of forming a reactive ester by a primary reaction between amino acids with a carboxyl group in the body, such as aspartic acid and glutamic acid, present on a skin surface, and a carbodiimide-based compound, and forming a covalent bond with a dye with an amine residue.

The following Reaction Scheme 2 shows a schematic diagram of a reaction of forming a reactive ester by a primary reaction between amino acids with a carboxyl group in the body, such as aspartic acid and glutamic acid, present on a dermal protein surface, substituting the reactive ester with a reaction accelerating additive to increase selectivity, and forming a covalent bond with a dye with an amine residue.

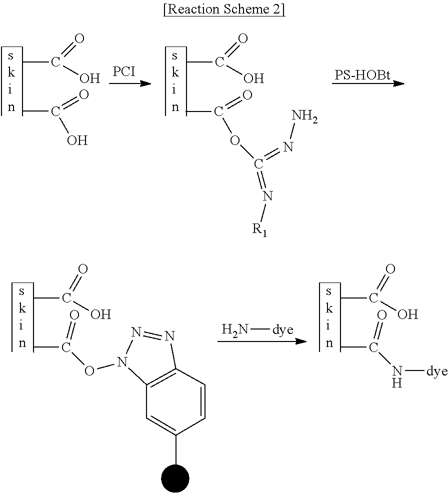

A dye adhered to a dermal protein by a peptide bond using a carbodiimide-based compound may be adhered to the skin semi-permanently without removal from the skin by a washing method with a shampoo, soap or a cleanser.

Due to the improved reaction efficiency between the dermal protein and the dye by adding the reaction accelerating additive to the carbodiimide-based compound, the dye adhered to the skin may not be easily dissociated in general washing with soap and may be almost permanently adhered to the skin.

This Reaction Scheme is a representative example for the skin and can also be applied to hair, fingernails, toenails, fiber or leather,

The present invention may also include a reactive dye in which a functional group forming covalent bonds with one or more proteins selected from the group consisting of carbodiimide, imidoester, arylazide, diazirine, hydroxymethyl phosphine, pentafluorophenylester, pyridyl disulfide, sulfo-hydroxysuccinimide ester, alkoxyamine, hydrazide, haloacetyl, azide, carbonate, aldehyde, propionaldehyde, butylaldehyde, nitrophenyl carbonate, aziridine, isocyanate, thiocyanate, epoxide, trecylate, succinimide, hydroxysuccinimidyl ester, imidazole, oxycarbonyl imidazole, imine, thiol, maleimide, vinylsulfone, ethyleneimide, thioether, acrylonitrile, acrylic or methacrylic ester, disulfide, ketone, carboxylic acids and phosphate is bound to the dye.

For example, as shown in Reaction Scheme 1, a dye prepared by a primary reaction of a carbodiimide-based compound and then substitution with a reaction accelerating additive may be included. In addition, the present invention may include a dyeing composition containing the dye substituted with the reaction accelerating additive.

In addition, by mixing with a fatty acid such as palmitic acid or stearic acid, a fatty alcohol, a cationic surfactant such as a linear or branched long-chain alkyl quaternary ammonium salt, a cationized polymer such as cationized cellulose, cationized guar gum or cationized polyvinylpyrrolidone, or silicone in order to additionally improve its effect, the dyeing composition according to the present invention may be easily prepared. In addition, for formulation as a cosmetic preparation, components for cosmetic preparation, such as a solvent, a surfactant, a thickening agent, a stabilizer, a preservative, a colorant, a pH adjuster, a metal ion sequestering agent, a pearling agent, an appearance improving agent, an emollient, a pigment, powder particles, may be additionally included. The components for preparation may be used at 40 to 99 parts by weight or 60 to 99 parts by weight with respect to 100 parts by weight of the total composition.

The dyeing composition according to the present invention may include a dye having a functional group to be used in formulation of a dyeing composition. Examples of hair products may include a pre-shampoo composition, a shampoo, a rinse, a treatment, a wax, a gel, a spray, a mousse, a hair lotion, a hair pack, a hair essence, a hair cream, a permanent hair dye, a temporary hair dye, a perming agent, a non-woven fabric and a sheet, and examples of skin products may include, generally, skin care products for smoothing and protecting skin (toner, serum, essence, lotion, cream, etc.), color cosmetics (make-up base, foundation, powder, eye shadow, lip gloss, lipstick, lip balm, etc.), finger nail and toe nail make-up (manicure, nourishing preparation, strengthening preparation, and top coat), cleansing cosmetics (foam cleanser, cleansing oil, cleansing lotion, cleansing cream, cleansing gel, pack, mask, soap, cleansing tissue, etc.), sun care cosmetics, and body cosmetics (body lotion, shower gel, body cream, body oil, etc.). Examples of fabric products may include fabric care products such as a fabric softener, a fabric dye, a laundry detergent, a treatment agent, a pre- or post-treatment agent, a laundry aiding agent, a partial stain remover and a spray. Examples of leather care products may include a cream, a lotion, an essence, a serum, a gel, a wax, a spray, a cleansing product, a cleanser, a partial stain remover, an ointment-type product, a temporary hair dye, a permanent hair dye, a polish, a strip and a sheet, but the present invention is not limited thereto.

More preferably, the dyeing composition according to the present invention includes a dye having a reactive functional group as well as a carbodiimide-based compound and a reaction accelerating additive. If the above-described preparation has lowered activity in an aqueous state, it is easier to maintain the activity of the preparation in a non-aqueous formulation, and a reaction may be induced by mixing with a buffer to adjust a pH immediately before use or contact with water during washing. Examples of non-aqueous formulations may include liquid-, sheet-, powder- and tablet-type formulations, oils, ampoules, and gels, which have been used as conventional non-aqueous cosmetic preparations. In addition, the dyeing composition according to the present invention may be formulated as an encapsulated single agent of the carbodiimide-based compound, the reaction accelerating additive and the dye, or as two separated agents of the carbodiimide-based compound and the reaction accelerating additive; and the dye. In addition, a method of blocking a derivative in the form in which a reactive functional group and a component having a dyeing function from moisture through encapsulation may also be used.

To improve a dyeing effect of the dyeing composition according to the present invention with a functional group, a dibasic acid ester, such as dioctyl succinate, dioctyl adipate or diethyl sebacate, a polyol, polyethylene glycol, propylene glycol, hexyl glycol, butanediol and an isomer thereof, hexylene glycol, butanediol and an isomer thereof, glycerol, benzyl alcohol, and ethoxy diglycol and a derivative thereof may be used. The above-described solvent increases the permeability of hair, skin, fingernails, toenails, fiber or leather, and is used as a solvent for an insoluble material. More preferably, solvents that are used to improve the activity retaining effect of a dye with a functional group include diethyl sebacate, ethoxy glycol, and bis-ethoxy glycol cyclohexane 1,4-dicarboxylate.

### EXAMPLES

Hereinafter, the present invention will be described in detail with reference to the following examples. The following examples are merely provided to exemplify the present invention, and the contents of the present invention are not limited to the following examples.

### Examples 1 and 2 and Comparative Examples 1 and 2

Lip gloss compositions of Examples 1 and 2 and Comparative Examples 1 and 2 were prepared with the compositions and contents shown in Table 1 below.

Water-phase components such as dipropylene glycol (DPG), a dye, 1,3-butylene glycol and glycerin, were heated to 70° C., and uniformly mixed for 5 minutes at 1500 rpm using a homomixer. Oil-phase components such as squalane, liquid paraffin, polybutene, lanolin, Vaseline and ceresin, which had been previously heated to 70° C., were added to the mixture of the water-phase components, and then uniformly mixed for 5 minutes at 3500 rpm using a homomixer. Subsequently, the temperature was decreased to 50° C., a counteractive, PCI, PS-HOBt, which is a reaction accelerating additive, were then added, and uniformly mixed for 3 minutes using a homomixer, followed by defoaming, filtration and cooling.

The hydroxybenzotriazole (PS-HOBt) was prepared by coupling a 10-μm bead-type polymer resin with hydroxybenzotriazole, wherein the polymer resin is in a form of 10-μm bead consisting of polystyrene represented by the following Formula 3 (molar ratio of m+n+x+y:z=99:1). The repeating unit in z below is a crosslinker which is not adhered to a specific location and randomly connects polymers, and synthesized with reference to Tetrahedron Letters 41 (2000) 2463-2466 and/or Chem. Rev. 2011, 111, 6557-6602.

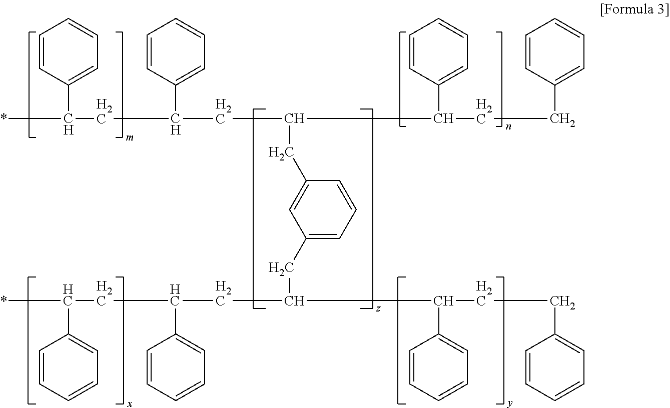

TABLE 1

| | | Comparative | | Comparative |
|---|---|---|---|---|
| Weight % | Example 1 | Example 1 | Example 2 | Example 2 |
| Dipropylene Glycol | To 100 | To 100 | To 100 | To 100 |
| (DPG) | | | | |
| Red No. 227 | 0.01 | 0.01 | — | — |
| Yellow No. 4 | — | — | 0.01 | 0.01 |
| Benzene, 1,3,-bis(1- | 0.1 | 0.1 | 0.1 | 0.1 |
| isocyanato-1- | | | | |
| methylethyl)-, | | | | |
| homopolymer, | | | | |
| polyethylene glycol | | | | |
| mono-me-ether- | | | | |
| blocked (PCI) | | | | |
| PS-hydroxybenzo- | 0.01 | — | 0.01 | — |
| triazole | | | | |
| 1,3 butylene glycol | 1.0 | 1.0 | 1.0 | 1.0 |
| Glycerin | 1.0 | 1.0 | 1.0 | 1.0 |
| Squalane | 1.0 | 1.0 | 1.0 | 1.0 |
| Liquid paraffin | 22.0 | 22.0 | 22.0 | 22.0 |
| Polybutene | 16.0 | 16.0 | 16.0 | 16.0 |
| Lanolin | 16.0 | 16.0 | 16.0 | 16.0 |
| Vaseline | 20.0 | 20.0 | 20.0 | 20.0 |
| Ceresin | 5.0 | 5.0 | 5.0 | 5.0 |

### Experimental Example 1

Each of the compositions of Examples 1 and 2 and Comparative Examples 1 to 3 was applied to three pig skin samples and treated for 30 minutes under constant temperature and humidity conditions (temperature: 32° C. and humidity: 50%) by a Franz cell experimental method. Afterward, to examine the dyeing effect on the pig skin surface, the skin was washed with a detergent solution. The degree of dyeing of the treated skin sample was confirmed by measuring a* values and b* values of a color difference meter (Konica Minolta CR-300 Chroma Meter), and the values were compared.

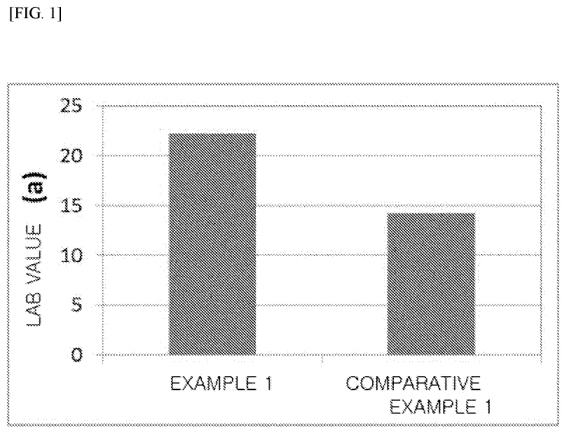

The results of the a* values of the color difference meter obtained as the experimental result are shown in Table 2 and FIG. 1 below. In addition, the results of the b* values are shown in Table 3 below.

TABLE 2

| | | Comparative |
|---|---|---|
| | Example 1 | Example 1 |
| a* value (average) | 22.21 | 14.34 |

TABLE 3

| | | Comparative |
|---|---|---|
| | Example 2 | Example 2 |
| b* value (average) | 35.54 | 20.42 |

As shown in Tables 2 and 3, when a reaction accelerating additive is added to PCI (Examples 1 and 2), it was confirmed that the dyeing effects of Red No. 227, which is an amine dye, and Yellow No. 4, which is a carboxy dye, caused by skin adhesion are significantly improved compared with when using only PCI (Comparative Examples 1 and 2). Therefore, various dyes as well as Red No. 227 and Yellow No. 4 can be adhered to the surface of skin, hair, finger nails, fiber or leather by the same method to remain for a long time, and it is expected that a cosmetic effect can be obtained consistently for a certain time.

1. A dyeing composition, comprising a carbodiimide-based compound, a reaction accelerating additive and a dye.

2. The dyeing composition of claim 1, wherein the reaction accelerating additive is one or more selected from the group consisting of hydroxysuccinimide, hydroxycarbamide, hydroxytriazole, hydroxybenzotriazole, hydroxypyridinone, hydroxyphenylbenzimidazole, hydroxyindolinone, hydroxybenzotriazine and oxyma.

3. The dyeing composition of claim 1, wherein the reaction accelerating additive is a water-soluble nonionic polymer, a bead- or resin-type polymer resin, or a type bound with silica beads.

4. The dyeing composition of claim 3, wherein the water-soluble nonionic polymer has a weight average molecular weight of 1,000 Da or more and less than 300,000 Da.

5. The dyeing composition of claim 3, wherein the water-soluble nonionic polymer is one or more polymers selected from the group consisting of polyethylene glycol, polyvinyl alcohol, polyethylene oxide, polyacrylonitrile, polyvinylpyrrolidone, polyisopropylacrylamide, a cellulose derivative, a starch derivative, dextran and guar gum.

6. The dyeing composition of claim 3, wherein the resin-type polymer has a weight average molecular weight of 1,000 Da or more and less than 300,000 Da.

7. The dyeing composition of claim 3, wherein the bead-type polymer resin has an average particle size of 100 nm to 1 mm.

8. The dyeing composition of claim 3, wherein the bead- or resin-type polymer resin is a polymer polymerized with a monomer selected from the group consisting of styrene, ethylene, butadiene, acrylonitrile, methyl styrene, terephthalate, ethylene chloride, ether ketone, ether imide, ether sulfone, phthalamide, phenylene ether, phenylene oxide, phenylene sulfide, sulfone, urethane, vinylidene fluoride and tetrafluoroethylene.

9. The dyeing composition of claim 3, wherein the silica bead has an average particle size of 100 nm to 1 mm.

10. The dyeing composition of claim 1, wherein the carbodiimide-based compound is contained at 0.001 to 10 parts by weight with respect to 100 parts by weight of the total composition.

11. The dyeing composition of claim 1, wherein the reaction accelerating additive, which forms peptide bonds, is contained at 0.0001 to 10 parts by weight with respect to 100 parts by weight of the total composition.

12. The dyeing composition of claim 1, wherein the dye contains a carboxyl or amine group in the molecule.

13. The dyeing composition of claim 1, wherein the dye is a natural or synthetic dye.

14. The dyeing composition of claim 13, wherein the natural dye is purpurin, munjistin, melanin or a melanin precursor.

15. The dyeing composition of claim 13, wherein the synthetic dye is one or more selected from the group consisting of Red No. 3, Red No. 104, Red No. 105, Red No. 201, Red No. 202, Red No. 220, Red No. 227, Red No. 230, Red No. 231, Red No. 232, Red No. 401, Red No. 405, Yellow No. 4, Yellow No. 202, Orange No. 207, Red No. 106, Red No. 213, Red No. 214, Red No. 215, Yellow No. 404, Yellow No. 405, Blue No. 403, Disperse Blue 1, Disperse Violet 1, Disperse Orange 3, Disperse Black 9, HC Blue 2, HC Red 3, HC Yellow 5 and HC Red 1.

16. The dyeing composition of claim 1, wherein the dye has a functional group which makes a covalent bond with a protein residue on the surface of hair, skin, fingernails, toenails, fiber or leather.

17. The dyeing composition of claim 16, wherein the functional group which makes a covalent bond with a protein residue is one or more selected from the group consisting of carbodiimide, imidoester, arylazide, diazirine, hydroxymethyl phosphine, pentafluorophenylester, tetrafluorophenylester, sulfodichlorophenolester, sulfonyl chloride, pyridyl disulfide, sulfo-hydroxysuccinimide ester, alkoxyamine, hydrazide, haloacetyl, azide, carbonate, aldehyde, propionaldehyde, butylaldehyde, nitrophenyl carbonate, aziridine, isocyanate, thiocyanate, epoxide, trecylate, succinimide, hydroxysuccinimidyl ester, imidazole, oxycarbonyl imidazole, imine, thiol, maleimide, vinylsulfone, ethyleneimide, thioether, acrylonitrile, acrylic or methacrylic ester, disulfide, ketone, carboxylic acid and phosphate.

18. The dyeing composition of claim 1, wherein the dye is contained at 0.001 to 50 parts by weight with respect to 100 parts by weight of the total composition.

19. The dyeing composition of claim 1, wherein the carbodiimide-based compound, the reaction accelerating additive and the dye are formulated as an encapsulated single agent, or the carbodiimide-based compound and the reaction accelerating additive; and the dye may be formulated as two separated agents.
